(19) United States
(12) Patent Application Publication
(10) Pub. No.: US 2022/0257487 A1
(43) Pub. Date: Aug. 18, 2022

(54) SULPHATE-FREE CONCENTRATED RHEO-FLUIDIFYING FOAMING COMPOSITION AND USES THEREOF

(71) Applicant: Laboratoires de Biologie Végétale Yves Rocher, La Gacilly (FR)

(72) Inventors: Marie-Agnés LAURENT, Brétigny-sur-Orge (FR); Pierre COLLIN, Chartres (FR)

(21) Appl. No.: 17/628,099

(22) PCT Filed: Jul. 9, 2020
(86) PCT No.: PCT/FR2020/051234

§ 371 Date: Jan. 18, 2022

(30) Foreign Application Priority Data
Jul. 18, 2019 (FR) F1908164

(51) Int. Cl. A61K 8/46; A61K 8/60; A61Q 19/10; C11D 1/28; C11D 1/66

(52) U.S. Cl. CPC A61K 8/466; A61K 8/604; C11D 1/662; C11D 1/28; A61Q 19/10

(57) ABSTRACT

The present invention relates to a surfactant composition comprising:

a) at least one anionic surfactant, and
b) at least one compound chosen from amphoteric surfactants and non-ionic surfactants,
wherein the total active material of said surfactants ranges from 25 to 70% by weight relative to the total weight of said composition,
wherein the anionic surfactant ratio is from 0.2 to 0.9, the amphoteric surfactant ratio is from 0.0 to 0.8 and the non-ionic surfactant ratio is from 0.0 to 0.8, said ratio being the ratio of the percentage by weight of active material of said surfactant in said composition to the percentage by weight of total active material of said surfactants in said composition,
the pH of the composition being between 4 and 9,
wherein said at least one anionic surfactant is chosen from the group consisting of alkyl sulfoacetate, isethionate and sodium lauroylsarcosinate.

The present invention also relates to a cosmetic, dermatological or detergent composition comprising the surfactant composition, as well as to a combination of a spray-generating device and the surfactant composition or the cosmetic composition, wherein said spray-generating device sprays said cosmetic composition.

### TECHNICAL FIELD

The present invention relates to a surfactant composition comprising at least one anionic surfactant and at least one compound chosen from amphoteric surfactants and nonionic surfactants, as well as to a cosmetic, dermatological or detergent composition comprising a surfactant composition of the invention.

The present invention has applications in particular in the field of cosmetics and cleaning in general.

### STATE OF THE ART

Not widely used twenty years ago, shower gel has gradually become an everyday body hygiene product because of the pleasant sensation it gives on the skin, the various parfums offered and its practicality of use.

Shower gels are usually packaged in bottles, possibly with pumps, or in flexible tubes. They are generally formulated to have a viscosity, being in the form of a gel, to enable them to be dispensed either by pouring from a bottle, by squeezing from a flexible tube or by squeezing from the pump of a pump bottle. The gel is usually diluted with water when applied to the skin, which also allows for the formation of a foam when the product is massaged or rubbed into the skin.

However, environmental concerns are becoming increasingly important in the cosmetic field. As a conventional shower gel contains at least 85% water, there is currently an effort to reduce the amount of water in this type of cosmetic product for ecological reasons, in order to reduce the amount of water transported on the one hand, and to reduce the amount of packaging on the other.

Thus, one way to reduce the amount of water in cosmetic products is to concentrate them.

There are concentrated personal care products on the market, although they are much less widespread than in the detergent sector (washing powder, concentrated dishwashing liquid, etc.).

In the 1990s, the Gühl brand offered a range of clear concentrated shampoos in bottles with service caps.

The limitation of these products is their application. Indeed, it is difficult to spread a very small quantity of product over a large surface, especially as the product's viscosity is high. This delays the dilution of the product with water and therefore the appearance of foam.

At the same time, there is another strong consumer expectation regarding shower gel products: indeed, they are demanding more and more naturalness in the products they use, especially when these are used daily and directly on the skin. In this respect, sulphates are currently the subject of controversy. Indeed, present in most foaming products such as shower gels, hair products and detergents, sulphates are surface-active agents that foam a lot and have a strong cleaning power. Thus, when used daily, certain sulphated surfactants are said to stimulate the production of sebum, promote blackheads, be irritating to the skin and scalp and accelerate the degradation of hair colouring. Consumers are therefore expecting more natural body care products, which are therefore more environmentally friendly, and which are better tolerated by a wider public.

There is therefore a real need for personal care products that overcome these defects, drawbacks and obstacles of the prior art, so as to improve their impact on the environment and human health.

### DESCRIPTION OF THE INVENTION

After extensive research, the applicant has succeeded in developing a personal care product, particularly for the skin and hair, which meets precisely these needs by providing a sulphate-free rheofluidifying composition concentrated in surfactants.

This composition has the property of fluidifying on spreading. The rheological properties of the composition according to the invention allow not only easy spreading on the skin and/or hair, but also dispensing of the composition by a spray-generating device, if desired by the user.

Advantageously, spray diffusion further improves the distribution of the composition on the body and the speed of dilution with water, to allow rapid foaming. On the other hand, the low viscosity of the formula allows a complete use of the concentrated product: little product remains in the plastic bottle at the end of use: the restitution rate is optimal.

Advantageously, the composition according to the invention is concentrated about 4 times in surfactants, that means in foaming active material, compared to a conventional shower gel. More largely between 3 and 5 and even more largely from 2 to 6.

The composition of the invention therefore has the advantage of being more environmentally friendly than existing gel-type personal hygiene products, since it can be presented in a smaller format.

In addition, the composition of the invention makes it possible to dispense with preservatives, for the benefit of consumer safety and to reduce the impact on the environment.

Because of its higher concentration than existing gel-type personal care products, the volume of products transported is divided by 4, which equally reduces CO₂ emissions during transport.

The reduction in manufactured volumes also reduces product storage capacity by a factor of 4.

As the packaging is reduced compared to a conventional product, the applicant estimates that the consumption of plastic in this context is reduced by 50% compared to a conventional gel-type product.

Because of its reduced size, the personal hygiene product resulting from the invention is more practical to use, as it is less heavy and more transportable. It can therefore be used more easily when travelling or when used outside the home as sporting activities.

In addition, it has the same advantages as a classic shower gel. In particular, it has a viscosity comparable to that of conventional foaming hygiene products and a great stability over time. In addition, the application of the hygiene product to wet skin or hair produces a pleasant foam, like that formed by a classic shower gel.

The applicant has shown that the rheo-fluidifying properties of the composition of the invention are linked to its physicochemical structure. Without wishing to be bound by a particular mechanism of action, the applicant has shown that the composition of the invention is a “structured” surfactant medium. In other words, the surfactants contained in the composition are organised in a lyotropic phase of the crystal-liquid type, also called mesophase. The phase is optically anisotropic and appears birefringent when observed under polarised light with an optical microscope. More precisely, it is a lamellar phase, where the surfactants are organised in bilayers, also called lamellae. The bilayers form a periodic stack, separated from each other by intermediate layers of water. Surfactant mixtures with lamellar phases are generally non-Newtonian liquids that can be pumped and have a cloudy appearance.

Furthermore, at the end of her research, the applicant has succeeded in obtaining these rheo-fluidification characteristics with sulphate-free surfactants, and which do not have the disadvantages of sulphated surfactants in terms of skin and scalp irritation.

Thus, a first object of the invention relates to a surfactant composition comprising:
a) at least one anionic surfactant, and
b) at least one compound chosen from amphoteric surfactants and non-ionic surfactants,
   wherein the total active material of said surfactants is between 25 and 70% by weight relative to the total weight of said composition,
   wherein the anionic surfactant ratio is between 0.2 and 0.9, the amphoteric surfactant ratio is between 0.0 and 0.8 and the non-ionic surfactant ratio is between 0.0 and 0.8, said ratio being the relationship between the percentage by weight of the active material of said surfactant in said composition and the percentage by weight of the total active material of said surfactants in said composition,
   the pH of the composition being between 4 and 9,
   wherein said at least one anionic surfactant is chosen from the group comprising an alkyl sulfoacetate, an isethionate and a sarcosinate.

Advantageously, the surfactant composition of the invention is free of sulphated surfactant. By “free of sulphated surfactant” or “sulphate-free composition”, within the meaning of the present invention, is meant that the surfactant composition of the invention is free, that means contains no sulphated surfactant. The surfactants thus referred to are in particular anionic surfactants derived from a sulphate, such as sodium lauryl sulphate (SLS), sodium laureth sulphate (SLES), ammonium lauryl sulphate (ALS) or ammonium laureth sulphate (ALES), and more generally surfactants comprising at least one sulphated group or a group which can be ionised to a sulphated group, in particular chosen from sulphate functions (—OSO₃H or —OSO₃⁻.

For the purposes of this invention, “active material” means the surfactant present in the commercial raw material containing the surfactant. The active material is thus the source of the surfactant properties of the raw material containing the surfactant. The raw material, usually distributed by a supplier, contains, in addition to the active material with the surfactant properties, other components, such as water or solvents, reaction residues and by-products. The percentage of active material in a raw material is obtained by subtracting the percentage of water or solvent reaction residues and by-products contained in the raw material to count only the percentage of surfactant.

“Total active material”, in the sense of the present invention, means the sum of the active material present in each raw material containing each surfactant, and included in the surfactant composition of the invention.

According to the invention, the total active material of the surfactants may be between 25 and 70% by weight relative to the total weight of the composition, for example between 25 and 35%, or between 30 and 55%, or between 35 and 45%, or between 30 and 35%, or between 35 and 40%, or between 30 and 40% or between 35 and 60%.

The composition of the invention has a pH between 4.0 and 9.0, inclusive. The pH may be, for example, between 4.5 and 8.5, or between 5.0 and 8.0, or between 5.5 and 8.5, or between 6.0 and 8.0, inclusive. Advantageously, the pH may be a physiological pH between 5.0 and 6.0, inclusive.

Advantageously, the surfactant composition of the invention has a good flow: the composition is fluid and flows by simply tilting the bottle: this is reflected by a measurement with a Brookfield rotary viscometer of type LV, of a viscosity advantageously <20,000 cps, or even less than 5,000 cps, or less than 3,000 cps, or even less than 2,500 cps, measured at speed 6 with the mobiles 2, 3 or 4 at the end of a measurement time of 1 min. This good flow is generally reflected in good rheofluidification, which can be observed during the measurement time: the viscosity decreases during the 1 min measurement time interval.

Advantageously, the surfactant composition of the invention has good sprayability. This can be reflected in a low high shear viscosity value and a low pour threshold value (for example with a pour threshold τ0<7 Pa), preferably with values close to 3 Pa.

The rheology of the surfactant composition can be measured by any method known to the skilled person, for example by stress flow measurement, using for example a Thermo Fisher RS600 rotary stress rheometer.

According to the invention, the surfactant composition may comprise at least one compound chosen from amphoteric surfactants and non-ionic surfactants. Thus, when the amphoteric surfactant ratio is 0.0, the non-ionic surfactant ratio cannot be equal to 0.0, so the value of 0.0 is excluded in this case. Conversely, when the non-ionic surfactant ratio is 0.0, the amphoteric surfactant ratio cannot be 0.0, so the value of 0.0 is excluded in this case.

According to the invention, the anionic surfactant ratio is between 0.2 and 0.9, for example between 0.3 and 0.8, or between 0.4 and 0.8, or between 0.4 and 0.6 or between 0.5 and 0.7.

According to the invention, the amphoteric surfactant ratio is between 0.0 and 0.8, for example between 0.1 and 0.6, or between 0.2 and 0.7, or between 0.2 and 0.5 or between 0.4 and 0.6.

According to the invention, the non-ionic surfactant ratio is between 0.0 and 0.8, for example between 0.01 and 0.25, or between 0.01 and 0.20, or between 0.05 and 0.1 or between 0.1 and 0.6, or between 0.2 and 0.7, or between 0.2 and 0.5.

For example, a surfactant composition according to the invention may consist of :
a) at least one anionic surfactant, and
b) at least one compound chosen from amphoteric surfactants and non-ionic surfactants,
   wherein the total active material of said surfactants is between 25 and 70% by weight relative to the total weight of said composition,
   wherein the anionic surfactant ratio is between 0.2 and 0.9, the amphoteric surfactant ratio is between 0.0 and 0.8 and the non-ionic surfactant ratio is between 0.0 and 0.8, said ratio being the ratio of the percentage by weight of active material of said surfactant in said composition to the percentage by weight of total active material of said surfactants in said composition,
   the pH of the composition being between 4 and 9,
   wherein said at least one anionic surfactant is chosen from the group comprising an alkyl sulfoacetate, an isethionate and a sarcosinate.

In this case, no compounds other than those indicated in this example are added to the composition of the invention.

According to another example, a surfactant composition according to the invention may comprise or consist of :
a) at least one anionic surfactant, and
b) at least one compound chosen from amphoteric surfactants and non-ionic surfactants,
   wherein the total active material of said surfactants is between 25 and 70% by weight relative to the total weight of said composition,
   wherein the anionic surfactant ratio is between 0.2 and 0.9, the amphoteric surfactant ratio is between 0.0 and 0.8 and the non-ionic surfactant ratio is between 0.0 and 0.8, said ratio being the ratio of the percentage by weight of active material of said surfactant in said composition to the percentage by weight of total active material of said surfactants in said composition,
   the pH of the composition being between 4 and 9,
   wherein said at least one anionic surfactant is chosen from the group comprising an alkyl sulfoacetate, an isethionate and a sarcosinate.

According to the invention, the ratios and percentages indicated above can be measured by any method known to the person skilled in the art. For example, for the determination of anionic surfactants, this may be the standard NF ISO 2271 (Determination of the content of anionic active substances by a manual or mechanical method by direct titration in two phases, January 1990, AFNOR).

Advantageously, the composition of the invention may comprise one, or two, or three of the said anionic surfactants. For example, the composition of the invention may contain at least one alone alkyl sulfoacetate as an anionic surfactant, or an alkyl sulfoacetate in combination with an isethionate and/or a sarcosinate. In another embodiment, the composition of the invention may contain an alone isethionate as an anionic surfactant, or an isethionate in combination with an alkyl sulfoacetate and/or a sarcosinate. In another embodiment, the composition of the invention may contain a sarcosinate alone as an anionic surfactant, or a sarcosinate in combination with an alkyl sulfoacetate and/or an isethionate.

According to the invention, the alkyl sulfoacetate may be chosen from sodium lauryl sulfoacetate, sodium coco sulfoacetate and myristyl sulfoacetate, and mixtures thereof. Preferably, the alkyl sulfoacetate is sodium lauryl sulfoacetate.

According to the invention, the sarcosinate may be chosen from sodium lauroyl sarcosinate, sodium myristoyl sarcosinate, potassium myristoyl sarcosinate, sodium cocoyl sarcosinate, sodium oleyl sarcosinate, ammonium oleyl sarcosinate and triethanolamine lauroyl sarcosinate, and mixtures thereof. Preferably, the sarcosinate is sodium lauroyl sarcosinate.

According to the invention, an isethionate may be chosen from the group comprising sodium cocoyl isethionate and sodium lauroyl methyl Isethionate and mixtures thereof. Preferably, the isethionate is sodium lauroyl methyl Isethionate.

Thus, the anionic surfactant may be at least one alkyl sulfoacetate. In this case, the composition of the invention may comprise an alkyl sulfoacetate and at least one anionic surfactant chosen from sodium lauroyl methyl isethionate and sodium lauroylsarcosinate. The composition of the invention may contain sodium lauryl sulfoacetate alone as an anionic surfactant, or sodium lauryl sulfoacetate in combination with an isethionate, in particular sodium lauroyl methyl isethionate and/or a sarcosinate, in particular sodium lauroylsarcosinate. For example, the composition may comprise two anionic surfactants such as sodium lauryl sulfoacetate and sodium lauroyl sarcosinate, optionally in combination with the substances and parameters provided in Example 7 below.

In another example, the at least one anionic surfactant may consist of an isethionate, in particular sodium lauroyl methyl isethionate, and a sarcosinate, in particular sodium lauroyl sarcosinate, optionally in combination with the substances and parameters provided for in Example 8 below.

In addition to the first group of anionic surfactants mentioned above, the composition of the invention may optionally comprise at least one other non-sulphated anionic surfactant different from those mentioned in the first group above, thus belonging to what may be termed the second group. Advantageously, the addition of at least one non-sulphated surfactant of the second group does not disturb the lamellar phase structure of the composition of the invention. Moreover, the addition of at least one non-sulphated surfactant of the second group may make it possible to improve the foaming quality of the composition of the invention, its rinsability or the stability of the composition, or the softness of the skin after application. This at least one anionic surfactant of the second group may be chosen from acylglutamates such as sodium lauroyl glutamate or sodium cocoyl glutamate, sulfosuccinates such as disodium cocopolyglucose sulfosuccinate, taurates such as sodium methyl cocoyl taurate or sodium stearoyl taurate, lactylates such as sodium stearoyl lactylate, sodium isostearyl lactylate or sodium cocoyl lactylate, carboxylates such as sodium lauryl glucose carboxylate or sodium laureth-13 carboxylate sulphonates such as sodium C14-16 olefin sulphonate, sodium C14-17 sec-Alkyl sulphonate and sodium xylene sulphonate, phosphoric acid derivatives, phosphates and alkyl phosphates such as dicetyl phosphate, C12-15 phosphate, Potassium cetyl phosphate or C9-15 alkyl phosphate, and alkyl polyglucoside derivatives such as sodium cocoglucoside tartrate, or disodium cocopolyglucose citrate.

According to the invention, the at least one amphoteric surfactant may be chosen from the group comprising N-alkylamidobetaines, betaines, sultaines, alkylpolyaminocarboxylates, alkylamphoacetates, derivatives thereof, and glycine derivatives.

According to the invention:
N-alkylamidobetaines can be chosen from the group comprising cocamidopropyl betaine and lauramidopropyl betaine and mixtures thereof;
betaines can be chosen from the group comprising coco betaine and lauryl betaine and mixtures thereof;
Sultaines can be cocamidopropyl hydroxysultaine,
the alkylpolyaminocarboxylates can be chosen from the group comprising sodium Carboxymethyl Tallow Polypropylamine and sodium Carboxymethyl Oleyl Polypropylamine and mixtures thereof;
the alkylamphoacetates may be chosen from the group comprising disodium cocoamphoacetate, sodium cocoamphoacetate and disodium lauroamphoacetate and mixtures thereof;
Glycine derivatives can be cocoamphopolycarboxyglycinate.

According to the invention, at least one non-ionic surfactant may be chosen from the group comprising glycolipids, alkypolyglucosides, glyceryl and fatty acid esters, sucrose and fatty acid esters, oxyalkylene sucrose esters, oxyalkylene glycerol esters, polyethylene glycol and fatty acid esters, sorbitan and fatty acid esters, polyglycerol fatty alcohols and glucamine derivatives.

According to the invention:
the glycolipids are chosen from the group comprising alkylpolyglucosides, sucrose esters, rhamnolipids, Mannosylerythritol lipids, sophorolipids and trehaloses;
the alkypolyglucosides may be chosen from the group comprising decyl glucoside, lauryl glucoside, caprylyl/capryl glucoside, and coco glucoside, and mixtures thereof;
the glyceryl and fatty acid esters may be chosen from glyceryl stearate, glyceryl ricinoleate and glyceryl oleate, and mixtures thereof;
the sucrose and fatty acid esters may be chosen from sucrose stearate, sucrose palm itate, sucrose laurate and sucrose distearate, and mixtures thereof;
The oxyalkylene sucrose esters may be chosen from methyl glucose caprate/caprylate/oleate, PEG-120 methyl glucose dioleate and PEG-20 methyl glucose sesquistearate, and mixtures thereof;
the oxyalkylene glycerol esters may be chosen from PEG-7 Glyceryl cocoate, PEG-80 Glyceryl cocoate, PEG-30 Glyceryl cocoate and PEG-200 hydrogenated glyceryl palmate, and mixtures thereof;
the polyethylene glycol and fatty acid esters can be chosen from the group PEG-8 stearate, PEG-20 stearate, PEG-40 stearate, PEG-50 stearate and PEG-100 stearate, and mixtures thereof;
the sorbitan and fatty acid esters may be chosen from the group comprising sorbitan palm itate, sorbitan stearate, sorbitan tristearate, sorbitan oleate and sorbitan trioleate, and mixtures thereof;
the glucamine derivatives may be chosen from the group comprising capryloyl or caproyl methyl glucamide, lauroyl methyl glucamide, lauroyl methyl glucamide and cocoyl methyl glucamide, and mixtures thereof.

Advantageously, whatever the anionic surfactant chosen, the amphoteric surfactant can be an N-alkylamidobetaine or an alkylamphoacetate, and the non-ionic surfactant can be an alkylpolyglucoside.

For example, whichever anionic surfactant is chosen, the amphoteric surfactant may be cocamidopropylbetaine or sodium cocoamphoacetate, and the non-ionic surfactant may be decylglucoside.

For example, in the surfactant composition of the invention, the anionic surfactant may be an isethionate, in particular sodium lauroyl methyl isethionate, the amphoteric surfactant may be cocamidopropyl betaine and the nonionic surfactant may be decylglucoside. Notably, in the surfactant composition of the invention, the anionic surfactant may consist of an isethionate, in particular sodium lauroyl methyl isethionate, the amphoteric surfactant may consist of cocamidopropyl betaine and the nonionic surfactant may consist of decylglucoside. Advantageously, the non-ionic surfactant ratio in such a composition may be between 0.01 and 0.8, for example between 0.01 and 0.25, or between 0.01 and 0.20, or between 0.01 and 0.5, or between 0.05 and 0.5, for example about 0.06.

In the course of their research, the inventors have also developed a solution allowing to limit the variability of the viscosity of surfactant compositions depending on the parfum used, especially at the time of preparation of the composition. This solution can be applied in cosmetic or dermatological applications, as well as in detergent applications, such as dishwashing liquids, laundry detergents, surface cleaners, for example floor cleaners or multi-purpose cleaners.

Thus, the invention further relates to a surfactant composition comprising :
a) at least one anionic surfactant,
b) at least one amphoteric surfactant, and
c) at least one non-ionic surfactant,
   wherein the total active material of said surfactants is between 25 and 70% by weight relative to the total weight of said composition,
   wherein the anionic surfactant ratio is between 0.20 and 0.90, the amphoteric surfactant ratio is between 0.01 and 0.80 and the non-ionic surfactant ratio is between 0.01 and 0.80, said ratio being the ratio of the percentage by weight of active material of said surfactant in said composition to the percentage by weight of total active material of said surfactants in said composition,
   the pH of the composition being between 4 and 9,
   wherein said at least one anionic surfactant is chosen from the group of isethionates.

Advantageously, the isethionate may consist of sodium lauroyl methyl isethionate, the amphoteric surfactant may consist of cocamidopropyl betaine and the nonionic surfactant may consist of decylglucoside.

Advantageously, the non-ionic surfactant ratio may be between 0.01 and 0.80, for example between 0.01 and 0.50, or between 0.01 and 0.25, or between 0.05 and 0.50, for example being about 0.06.

The anionic surfactant ratio is between 0.2 and 0.9, for example between 0.3 and 0.8, or between 0.4 and 0.8, or between 0.4 and 0.6 or between 0.5 and 0.7.

The amphoteric surfactant ratio is between 0.01 and 0.80, for example between 0.10 and 0.60, or between 0.20 and 0.70, or between 0.20 and 0.50 or between 0.40 and 0.60.

Whatever the composition of the invention as defined above, the latter may also comprise at least one perfume. By “perfume”, in the sense of the present invention, is meant a perfuming raw material or a mixture of perfuming raw materials, also called “perfume concentrate”, of natural and/or synthetic origin, solubilised or not in a solvent for perfumery raw materials. In other words, it can be any odorous composition or any mixture of odorous raw materials, presenting olfactory characteristics compatible with a use in cosmetics, generally highly concentrated, generally proposed packaged by a perfumer. The solvent may be any suitable solvent known to the skilled person, such as, for example, ethanol, Dipropylene glycol, triethylcitrate, isopropyl myristate, triacetin, this list not being restrictive. The raw materials of natural origin can be any suitable raw material known to the person skilled in the art, for example essential oils, concretes, absolutes or plant extracts. The proportions between solvent and odorous raw materials can be very variable according to the olfactory power of the odorous raw materials and their solubility, according to what is classically achieved in the field of perfumes, known to the person skilled in the art. According to the invention, the composition can thus additionally comprise perfume whose percentage by weight in the composition can range from 0.0 to 10% by weight relative to the total weight of said composition, the limits being included, for example from 0.05 to 10%, or from 0.1 to 10%, or from 0.5 to 10%, or from 0.5 to 5.0%, or from 1 to 10%, or from 2 to 10%, or from 3 to 10%, or from 5 to 10%. The perfume may be any commercial perfume composition, for example Perfume Lemon Basil (Givaudan) or Perfume Acidulated Berry (Robertet). Advantageously, the perfume may lead to lowering the viscosity of the surfactant mixture to obtain a more fluid product, and/or to lower the flow threshold of the composition, thus improving sprayability.

In one embodiment, regardless of the surfactants chosen, the surfactant composition may be free of sodium chloride other than that which may be provided by the surfactants. In this case, no sodium chloride as such is added during the preparation of the surfactant composition of the invention. However, the surfactant compositions forming part of the composition may contain it, which implies that a small amount of sodium chloride may possibly be detected, particularly in trace amounts, in the surfactant composition of the invention. This may be, for example, from about 0.0 to about 6.0% by weight of sodium chloride from the surfactant compositions, based on the total weight of said composition, for example between 0.001 and 6.0%, or 0.005 and 1.0%, or 0.01 and 1.0%, or 0.05 and 1.0%, or 0.05 and 0.1%. The absence of added sodium chloride advantageously confers a better safety of the composition compared to conventional products, in which sodium chloride is added.

In another embodiment, regardless of the surfactants chosen, the composition of the invention may further comprise from 0.0 to 8.0% of sodium chloride other than that which may be provided by the surfactants, the value of 0.0% being excluded. For example, the composition of the invention may comprise from about 0.001 to 8.0%, or from about 0.1 to 4.0%, or from about 0.5 to 4.0%, or from about 1.0 to 3.0, or from about 1.0 to 4.0%, or from about 1.0 to 6.5%, or from about 2.0 to 6.0%. For example, the composition of the invention may comprise 4.0%, or 5.0%, or 6.0%, or 7.0%, or 8.0% of sodium chloride other than that which may be provided by the surfactants.

The surfactant composition according to the invention may in particular constitute a composition for cutaneous application, used in particular in the cosmetic or dermatological fields, as cleansing products for the skin, including the body and the face, the scalp and/or the hair. A composition for topical application may optionally contain a physiologically acceptable medium, that means compatible with the skin, the mucous membranes, the scalp and/or the hair. More particularly, it may be a skin cleansing composition.

According to the invention, the surfactant composition may be in a form chosen from a shower gel, a facial skin cleansing gel, a shampoo and a hand washing gel.

According to the invention, the surfactant composition of the invention may be presented in a package chosen from a bottle, for example of the type provided with a cap or a pump (dosing or spray), a tube, for example a flexible tube and a jar.

Advantageously, the surfactant composition of the invention does not comprise a preservative, this being conducive to better safety.

A further object of the invention relates to a cosmetic or dermatological composition comprising a surfactant composition according to the invention.

Another object of the invention relates to a detergent composition for cleaning surfaces, comprising a surfactant composition as defined above. This detergent composition may be in a form chosen from a laundry detergent, a dishwashing liquid, in particular a concentrate, and a surface cleaner, for example a floor cleaner or a multi-purpose cleaner.

According to an embodiment, the cosmetic, dermatological or detergent composition may consist of, that means comprise exclusively, the surfactant composition of the invention.

Advantageously, the cosmetic, dermatological or detergent composition of the invention has the same rheo-fluidizing properties as well as the other advantageous properties as the surfactant composition of the invention.

According to the invention, the cosmetic composition may comprise a cosmetically acceptable vehicle.

In the present invention, “cosmetic composition” is taken to mean any composition with cosmetic, that means aesthetic, purposes which can be brought into contact with the superficial parts of the human body, for example the epidermis, the hair and capillary systems, the external organs and the external mucous membrane. Advantageously, a cosmetic composition makes it possible, exclusively or mainly, to clean, protect or maintain them in good condition.

In this document, “dermatological composition” means any composition for dermatological purposes, that means a composition which can be brought into contact with the superficial parts of the human body, for treatment of the skin, mucous membranes and skin appendage, nails, hair.

“Cosmetically or dermatologically acceptable vehicle” means a vehicle suitable for use in contact with human and animal skin cells, in particular epidermal cells, without undue toxicity, irritation, allergic response and the like, and commensurate with a reasonable benefit/risk ratio. The vehicle is used in proportions compatible with the high concentration of the composition of the invention.

According to the invention, the cosmetic composition of the invention may also comprise adjuvants usually used in the cosmetic field, chosen from oils, plant butters, plant or synthetic waxes, active materials, perfumes, preservatives, sequestrants (EDTA or sodium phytate, for example), antioxidants, cationic polymers and cationic surfactants, pearlescent and opacifying agents, pigments, mineral or organic fillers such as talc, kaolin, starch, exfoliants (solid vegetable, mineral or synthetic particles with an abrasive effect on the skin), pearlescents and flakes, dyes, sun filters, acids and bases for pH adjustment, synthetic or natural gelling polymers. The quantities of these various adjuvants are those conventionally used in the field in question, and for example from 0.01 to 20% of the total weight of the composition. These additives and their concentrations must be such that they do not modify the property sought for the composition of the invention.

Examples of oils include vegetable oils such as jojoba, avocado, sesame, sunflower, rapeseed, corn, soybean, safflower, and grape seed, or mineral oils such as paraffin oils, or synthetic oils such as isopropyl myristate, cococaprylate caprate, ethylhexyl palmitate and alkyl benzoate, or volatile or non-volatile silicone oils such as polydimethylsiloxanes (PDMS) and cyclodimethylsiloxanes or cyclomethicones, or fluorinated or fluorosilicone oils, as well as mixtures of these oils The amount of oil must not alter the desired property of the composition of the invention.

Examples of active materials include for example moisturisers and for example polyols such as glycerine, glycols, polyethylene glycols and sugars and their derivatives, natural extracts, piroctone olamine, zinc pyrithione, salicylic acid, urea, vitamins and their derivatives (for example D panthenol or tocopherol acetate), protein hydrolysates.

The composition of the invention may be obtained by any suitable method known to the person skilled in the art for the manufacture of a foaming cosmetic composition. It may be, for example, a simple mixture.

Advantageously, the composition can be a skin composition, and can be intended to be rinsed or not rinsed.

According to the invention, the cosmetic composition may be in a form chosen from shower gel, facial skin cleansing gel, shampoo and hand washing gel.

A further object of the invention relates to a cosmetic use of the cosmetic composition according to the invention, for cleaning the skin and/or the skin appendages and/or the scalp.

A further object of the invention relates to a non-therapeutic cosmetic treatment method comprising applying to the skin and/or the skin appendages and/or the scalp a surfactant composition of the invention or a cosmetic composition of the invention.

Advantageously, the cosmetic treatment method may be a method for cleansing residual soiling of human keratinous materials (also cleansing of sweat and lipids), wherein the cosmetic composition of the invention is applied to the skin and/or the skin appendages and/or the scalp in the presence of water. Advantageously, the application may comprise a massage to form a foam, which may be removed together with the dirt residue by rinsing with water.

In the context of the cosmetic methodes according to the invention, or of the use according to the invention, the use is understood to be a non-therapeutic use, for example for the treatment of healthy skin, that means skin not showing a pathological condition. It may also be skin which does not show any visible or perceptible trace of an external aggression, such as itching, sunburn, burns, stings, signs of inflammation, wounds, this list not being restrictive.

Preferably, any cosmetic use and any cosmetic method according to the invention are non-therapeutic cosmetic uses and non-therapeutic cosmetic methods respectively.

A further object of the invention relates to a combination of a spray-generating device or a device for delivering a dose and a surfactant composition or a cosmetic composition according to the invention, wherein the spray-generating device diffuses the cosmetic composition or the surfactant composition as a spray.

The device for delivering a dose can be for example a valve capsule, for example a valve capsule of the type Capsule Double Invio—ref V143-145 marketed by the Aptar Group supplier Seaquist.

Advantageously, the surfactant composition of the invention having a viscosity comparable to conventional shower gels but being rheofluidizing, is sprayable, that means it can be dispensed by means of a spray-generating device. Without wishing to be bound by an explanation of a mechanism of action, the Applicant hypothesises that the organisation of the surfactants of the surfactant composition into lamellar phases allows sprayability.

Advantageously, the distribution by means of a spray-generating device of the cosmetic composition or surfactant composition according to the invention facilitates the distribution of the composition on the keratinous materials, in particular facilitates the distribution of a small amount of composition (insofar as the formula is highly concentrated) over a large surface.

Advantageously, the surfactant composition of the invention being rheo-fluidifying, it allows, in combination with distribution by spray, the easy distribution of a lower dose of product, for example 4 times lower, than with a conventional shower gel, for a surface area of keratinous material, in particular of skin, which is identical to or greater than that covered by a greater quantity of conventional product.

The spray-generating device can be any device available on the market, for example a dosing bottle with a spray pump, for example the PZ2 190 DLD1 Helios Pump (Aptar).

Further advantages may become apparent to the person skilled in the art from the examples below.

### EXAMPLES

### Example 1: Method for the Preparation of a Surfactant Composition

Cocamidopropylbetaine (amphoteric surfactant, referred to in Table I as “CAPB”) and decylglucoside (non-ionic surfactant) are added to water at room temperature (approximately 20° C.). Citric acid is then added to the mixture at room temperature. Perfume is then added to the mixture, followed by sodium lauryl sulfoacetate (anionic surfactant referred to in Table I as “LSaNa”), and optionally sodium chloride; the whole is mixed at 65° C.

The proportions of the different components are those given in Table I below; the ratio values are noted in the following order: anionic/am photeric/non-ionic.

TABLES 1

| | | | | | | Commercial |
|---|---|---|---|---|---|---|
| | | | | Commercial | Commercial | raw materials, |
| | | | | raw materials, | raw materials, | decyl glucoside |
| % active | | Nacl | | LSaNa (Lathanol | CAPB (Tegobetain | (Plantacare |
| material | pH | %. | Comment | LAL Stepan) | F50 EVONIK) | 2000 UP BASF) |
| 40 | 7.63 | 0 | Oily streaks | 25.0-30.0 | 35.0-40.0 | 0.0-1.0 |
| 40 | 7.91 | 0 | Oily streaks | 20.0-25.0 | 20.0-25.0 | 30.0-35.0 |
| 40 | 5 | 0 | Structured | 20.0-25.0 | 20.0-25.0 | 30.0-35.0 |
| | | | environment | | | |
| 40 | 5 | 4 | Structured | 20.0-25.0 | 20.0-25.0 | 30.0-35.0 |
| | | | environment | | | |
| 40 | 6 | 0 | Oily streaks | 20.0-25.0 | 48.0-52.0 | 11.0-16.0 |
| 40 | 5 | 0 | Oily streaks | 20.0-25.0 | 48.0-52.0 | 11.0-16.0 |
| 40 | 5 | 4 | Oily streaks | 20.0-25.0 | 48.0-52.0 | 11.0-16.0 |
| 40 | 7 | 0 | Structured | 20.0-25.0 | 20.0-25.0 | 30.0-35.0 |
| | | | environment | | | |
| 40 | 7 | 4 | Oily streaks | 20.0-25.0 | 20.0-25.0 | 30.0-35.0 |
| 40 | 7 | 0 | Oily streaks | 11.0-16.0 | 40.0-45.0 | 25.0-30.0 |
| 40 | 7 | 0 | Oily streaks | 20.0-25.0 | 47.0-52.0 | 11.0-16.0 |
| 40 | 7 | 4 | Oily streaks | 20.0-25.0 | 47.0-52.0 | 11.0-16.0 |
| 30 | 5 | 4 | Structured | 14.0-19.0 | 35.0-40.0 | 8.0-13.0 |
| | | | environment | | | |
| 30 | 5 | 0 | Oily streaks | 20.0-25.0 | 20.0-25.0 | 11.0-16.0 |
| 30 | 5 | 0 | Oily streaks | 20.0-25.0 | 40.0-45.0 | 0.0-1.0 |
| 30 | 5 | 4 | Oily streaks | 20.0-25.0 | 40.0-45.0 | 0.0-1.0 |
| 30 | 7 | 4 | Structured | 13.0-18.0 | 35.0-40.0 | 8.0-13.0 |
| | | | environment | | | |
| 30 | 7 | 0 | Structured | 20.0-25.0 | 20.0-25.0 | 12.0-16.0 |
| | | | environment | | | |
| 30 | 7 | 0 | Structured | 20.0-25.0 | 40.0-45.0 | 0.0-1.0 |
| | | | environment | | | |
| 30 | 7 | 4 | Oily streaks | 20.0-25.0 | 40.0-45.0 | 0.0-1.0 |

### Example 2: Method for the Preparation of a Surfactant Composition

Cocamidopropylbetaine (amphoteric surfactant, referred to in Table I as “CP betaine”) and decylglucoside (non-ionic surfactant) are added to water at room temperature (approximately 20° C.). Citric acid is then added to the mixture at room temperature. Perfume is then added to the mixture, followed by sodium lauroyl methyl isethionate (anionic surfactant), and optionally sodium chloride; the whole is mixed at 65° C.

The proportions of the different components are those given in Table II below; the ratio values are noted in the following order: anionic/amphoteric/non-ionic.

TABLE 2

| | | | | | Commercial | Commercial |
|---|---|---|---|---|---|---|
| | | | | Commercial | raw materials, | raw material |
| | | | | raw materials, | CAPB (Empigen | decyl glucoside |
| | | Nacl | | ISELUX | BS/H50/MB | (Plantacare |
| % MA | pH | %. | Comment | Innospec | Innospec) | 2000UP BASF) |
| 40 | 6.2 | 0 | Oily streaks | 21.0-26.0 | 52.0-57.0 | 0.0-1.0 |
| 40 | 6.8 | 0 | Oily streaks | 15.0-20.0 | 47.0-52.0 | 13.0-18.0 |
| 40 | 5 | 0 | Oily streaks | 21.0-26.0 | 52.0-57.0 | 0.0-1.0 |
| 40 | 5 | 0 | Oily streaks | 15.0-20.0 | 47.0-52.0 | 12.0-16.0 |
| 40 | 5 | 0 | Oily streaks | 23.0-28.0 | 25.0-30.0 | 15.0-20.0 |
| 40 | 5 | 4 | Oily streaks | 15.0-20.0 | 47.0-52.0 | 12.0-16.0 |
| 40 | 5 | 4 | Oily streaks | 23.0-28.0 | 25.0-30.0 | 17.0-22.0 |
| 40 | 5 | 8 | Oily streaks | 23.0-28.0 | 53.0-58.0 | 0.0-1.0 |
| 40 | 5 | 8 | Oily streaks | 15.0-20.0 | 47.0-52.0 | 12.0-16.0 |
| 40 | 5 | 8 | Oily streaks | 15.0-20.0 | 20.0-25.0 | 30.0-35.0 |
| 40 | 7 | 0 | Oily streaks | 23.0-28.0 | 53.0-58.0 | 0.0-1.0 |
| 40 | 7 | 0 | Structured | 15.0-20.0 | 47.0-52.0 | 12.0-16.0 |
| | | | environment | | | |
| 40 | 7 | 4 | Oily streaks | 23.0-28.0 | 53.0-58.0 | 0.0-1.0 |
| 40 | 7 | 4 | Oily streaks | 15.0-20.0 | 47.0-52.0 | 12.0-16.0 |
| 40 | 7 | 8 | Dense | 23.0-28.0 | 53.0-58.0 | 0.0-1.0 |
| | | | network of | | | |
| | | | oily streaks | | | |
| 40 | 7 | 8 | Oily streaks | 15.0-20.0 | 47.0-52.0 | 12.0-16.0 |
| 40 | 7 | 8 | Oily streaks | 23.0-28.0 | 25.0-30.0 | 17.0-21.0 |
| 30 | 5 | 0 | Oily streaks | 15.0-20.0 | 40.0-45.0 | 0.0-1.0 |
| 30 | 5 | 4 | Oily streaks | 15.0-20.0 | 40.0-45.0 | 0.0-1.0 |
| 30 | 5 | 8 | Oily streaks | 12.0-16.0 | 35.0-40.0 | 8.0-12.0 |
| 30 | 5 | 8 | Oily streaks | 15.0-20.0 | 20.0-25.0 | 12.0-16.0 |
| 30 | 7 | 4 | Oily streaks | 15.0-20.0 | 40.0-45.0 | 0.0-1.0 |
| 30 | 7 | 8 | Oily streaks | 15.0-20.0 | 40.0-45.0 | 0.0-1.0 |

### Example 3: Method for the Preparation of a Surfactant Composition

Cocamidopropylbetaine (amphoteric surfactant) and decylglucoside (non-ionic surfactant) are added to water at room temperature (about 20° C.). Citric acid is then added to the mixture at room temperature. Perfume is then added to the mixture, followed by sodium lauroylsarcosinate (anionic surfactant), and optionally sodium chloride, all mixed at room temperature.

The proportions of the different components are those given in Table III below; the ratio values are noted in the following order: anionic/amphoteric/non-ionic.

TABLE 3

| | | Commercial | Commercial raw | Commercial raw |
|---|---|---|---|---|
| | | raw material | material CPB % | material |
| | | sodium lauroyl | (Empigen | Decylglucoside % |
| Total active | | sarcosinate | BS/H50/MB | (Plantacare 2000UP |
| material | comment | eversoft S12%. | Innospec) | BASF) |
| 32.3 | structured | 50.0-55.0 | 45.0-50.0 | 0.0-1.0 |
| | environment | | | |
| 34 | structured | 20.0-25.0 | 75.0-80.0 | 0.0-1.0 |
| | environment | | | |
| 35 | structured | 40.0-45.0 | 10.0-15.0 | 41.0-46.0 |
| | environment | | | |
| 36 | structured | 25.0-30.0 | 10.0-15.0 | 58.0-62.0 |
| | environment | | | |
| 38 | structured | 13.0-18.0 | 18.0-23.0 | 60.0-65.0 |
| | environment | | | |
| 42.7 | structured | 15.0-20.0 | 45.0-50.0 | 33.0-38.0 |
| | environment | | | |
| 38.54 | structured | 28.0-33.0 | 25.0-30.0 | 40.0-45.0 |
| | environment | | | |
| Max: 42.7 | | | | |
| Min: 32.3 | | | | |

### Example 4: Examples of Compositions of the Invention Containing Sodium Lauryl Sulfoacetate as an Anionic Surfactant

Examples of the surfactant composition of the invention are given in Table IV; the ratio values are noted in the following order: anionic/amphoteric/nonionic.

TABLE 4

| INCI name (trade name) | % in the composition | % in the composition | % in the Composition |
|---|---|---|---|
| SODIUM LAURYL | 20.0-25.0 | 20.0-25.0 | 20.0-25.0 |
| SULFOACETATE (Lathanol | | | |
| LAL Stepan) | | | |
| COCAMIDOPROPYL | 48.0-52.0 | 20.0-25.0 | 40.0-45.0 |
| BETAINE (TEGO BETAIN | | | |
| F50) | | | |
| DECYL GLUCOSIDE | 12.0-16.0 | 30.0-35.0 | 0.0-1.0 |
| (PLANTACARE 2000 UP | | | |
| BASF) | | | |
| CITRIC ACID | 1.0-4.0 | 1.0-2.0 | 0.0-1.0 |
| NaCl | 0.0-1.0 | 2.0-5.0 | 0.0-1.0 |
| water | QSP100 | QSP100 | QSP100 |
| Total surfactant | 40 | 40 | 30 |
| pH | 5 | 7 | 5 |
| Aspect | very slight disorder | very slight disorder | very slight disorder |
| Bright microscopic | Oily streaks | Oily streaks | Oily streaks |
| appearance X10 Polarised | | | |
| light | | | |
| Rheofluidification during | yes | yes | yes |
| viscosity measurement | | | |

Example 5: Examples of Compositions of the Invention Containing Sodium Lauroyl Methyl Isethionate as an Anionic Surfactant

Examples of the surfactant composition of the invention are given in Table V; the ratio values are noted in the following order: anionic/amphoteric/nonionic.

TABLE 5

| INCI name (trade name) | % in the composition | % in the composition | % in the composition |
|---|---|---|---|
| SODIUM LAUROYL | 21.0-26.0 | 15.0-20.0 | 15.0-20.0 |
| METHYL ISETHIONATE | | | |
| (ISELUX, Innospec) | | | |
| COCAMIDOPROPYLBETAIN | 53.0-58.0 | 48.0-52.0 | 40.0-45.0 |
| (EMPIGEN BS/H50/MB | | | |
| Innospec) | | | |
| DECYL GLUCOSIDE | 0.0-1.0 | 11.0-16.0 | 0.0-1.0 |
| (PLANTACARE 2000 UP | | | |
| BASF) | | | |
| CITRIC ACID | 0.1-0.4 | 0.2-0.6 | 0.1-0.5 |
| NaCl | 0.0-0.1 | 0.0-0.1 | 0.0-0.1 |
| water | QSP100 | QSP100 | QSP100 |
| Total surfactant | 40 | 40 | 30 |
| pH | 5 | 5 | 4.9 |
| Aspect | very slight disorder | very slight disorder | very slight disorder |
| Bright microscopic | Oily streaks | Oily streaks | Oily streaks |
| appearance X10 Polarised | | | |
| light | | | |
| Rheofluidification during | yes | yes | yes |
| viscosity measurement | | | |

### Example 6: Examples of Compositions of the Invention Containing Sodium Lauroyl Sarcosinate as an Anionic Surfactant

Examples of the surfactant composition of the invention are given in Table VI; the ratio values are noted in the following order: anionic/amphoteric/nonionic.

TABLE 6

| INCI name (trade name) | % in the composition | % in the composition |
|---|---|---|
| SODIUM LAUROYL SARCOSINATE | 50.0-55.0 | 20.0-25.0 |
| (EVERSOFT S12, Sino Lion) | | |
| COCAMIDOPROPYLBETAIN EMPIGEN | 45.0-50.0 | 75.0-80.0 |
| BS/H50/MB Innospec) | | |
| DECYL GLUCOSIDE (PLANTACARE | 0.0 | 0.0 |
| 2000 UP BASF) | | |
| CITRIC ACID | 0.0 | 0.0 |
| NaCl | 0.0 | 0.0 |
| water | 0 | 0 |
| Total surfactant | 30.0-35.0 | 32.0-36.0 |
| pH | 7 | 6.4 |
| Aspect | very slight disorder | very slight disorder |
| Bright microscopic appearance X10 | brushed metal look | brushed metal look |
| Polarised light | | |
| Rheofluidification during viscosity | yes | yes |
| measurement | | |

### Example 7: Examples of Compositions of the Invention Containing Sodium Lauryl Sulfoacetate and Sodium Lauroyl Sarcosinate as Anionic Surfactants

Examples of the surfactant composition of the invention are given in Table VII.

TABLE 7

| INCI name | Percentage | Percentage | Percentage |
|---|---|---|---|
| SODIUM LAURYL SULFOACETATE | 9.0-12.0 | 9.0-12.0 | 9.0-12.0 |
| (LATHANOL LAL STEPAN) | | | |
| SODIUM LAUROYL SARCOSINATE | 10.0-13.0 | 10.0-13.0 | 10.0-13.0 |
| (EVERSOFT S12 Sino Lion) | | | |
| COCAMIDOPROPYLBETAIN | 60.0-65.0 | 46.0-51.0 | 58.0-62.0 |
| (EMPIGEN BS/H50/MB Innospec) | | | |
| DECYL GLUCOSIDE (PLANTACARE | 5.0-9.0 | 15.0-19.0 | 0.0 |
| 2000 UP BASF) | | | |
| CITRIC ACID | 1.0-2.0 | 0.1-1.0 | 0.0 |
| NACL | 0.0 | 1.0-3.0 | 0.0 |
| water | QSP | QSP | QSP |
| Total surfactant | 35.0-40.0 | 35.0-40.0 | 30.0-35.0 |
| pH | 5.7 | 6.7 | 5.7 |
| Aspect | very slight | very slight | very slight |
| | disorder | disorder | disorder |
| Bright microscopic appearance | Not tested | Not tested | Not tested |
| X10 Polarised light | | | |
| Rheofluidification during viscosity | yes | yes | yes |
| measurement | | | |

### Example 8: Examples of Compositions of the Invention Containing Sodium Lauroyl Methyl Isethionate and Sodium Lauroyl Sarcosinate as Anionic Surfactants

Examples of the surfactant composition of the invention are given in Table VIII.

TABLE 8

| INCI name (trade name) | Percentage | Percentage |
|---|---|---|
| SODIUM LAUROYL METHYL | 8.0-12.0 | 8.0-12.0 |
| ISETHIONATE (ISELUX, Innospec°) | | |
| SODIUM LAUROYL SARCOSINATE | 25.0-29.0 | 25.0-29.0 |
| (EVERSOFT S12 Sino Lion) | | |
| COCAMIDOPROPYLBETAIN (EMPIGEN | 43.0-46.0 | 46.0-50.0 |
| BS/H50/MB, Innospec) | | |
| DECYL GLUCOSIDE (PLANTACARE | 0.0 | 5.0-9.0 |
| 2000 UP, BASF) | | |
| CITRIC ACID | 0.2 | 0.2 |
| NaCl | 0.0 | 0.0 |
| water | QSP | QSP |
| Total surfactant | 30.0-34.0 | 35.0-38.0 |
| pH | 6.0 | 6.0 |
| Aspect | very slight | very slight |
| | disorder | disorder |
| Bright microscopic appearance X10 | Not tested | Not tested |
| Polarised light | | |
| Rheofluidification during viscosity | yes | yes |
| measurement | | |

### Example 9: Example of a Composition of the Invention Containing Sodium Lauroyl Methyl Isethionate as an Anionic Surfactant

An example of a surfactant composition of the invention is given in Table 9.

TABLE 9

| | | | Active | | | |
|---|---|---|---|---|---|---|
| | | | material | Active | | |
| Phase | Description | Quantity | MP | material | ratios | Common Name |
| A | DECYLGLUCOSIDE 2000 | 5 | 53 | 2.7 | 0.063 | DECYL GLUCOSIDE |
| | UP-AMBAL°°° | | | | | & AQUA |
| A | ISELUX | 24.3 | 82 | 19.9 | 0.474 | Sodium Lauroyl |
| | | | | | | Methyl Isethionate |
| A | COCAMIDOPROPYLBETAINE | 55.5 | 35 | 19.4 | 0.462 | COCAMIDOPROPYL |
| | F50-MBAL | | | | | BETAINE & AQUA |
| A | PURIFIED WATER | 12.98 | | | | AQUA |
| A | CITRIC ACID | 0.12 | | | | CITRIC ACID |
| | MONOHYDRATE | | | | | |
| B | POMEGRANATE PINK | 2 | | | | PERFUM |
| | BERRIES G116 32882 | | | | | |
| C | GRENADE GLY75 | 0.1 | | | | GLYCERIN & AQUA |
| | (organic plant) | | | | | & PUNICA |
| | | | | | | GRANATUM FRUIT |
| | | | | | | EXTRACT |
| | | | Total MA | 42.0 | | |
| | | | in formula | | | |

The composition as described in Table 9 has the advantage of showing a regulated viscosity, which remains below 2500 cps, whatever the parfum tested (mango, coconut, vanilla and pomegranate).

By way of example, a comparison between the viscosity measured for the composition in Table 9, and the viscosity measured for the same composition in which decylglucoside is replaced by glycerine in the same proportion in the composition (referred to as the “5% glycerine comparison”), is given in Table 10.

Viscosities are measured with a Brookfield rotary viscometer type LVT speed 6, mobile 2, 3 or 4 depending on the viscosity range: 2000cps (mobile 2) 6000cps (mobile 3) 18000cps (mobile 4).

TABLE 10

| | Vanilla | Coconut | Mango | Pomegranate |
|---|---|---|---|---|
| | parfum | parfum | parfum | parfum |
| Composition of | 1850 cps | 1935 cps | 2140 cps | 1735 cps |
| Table 9 | | | | |
| Comparative 5% | 1560 cps | 2005 cps | 14600 cps | 8000 cps |
| glycerine | | | | |

The viscosity measured for the composition “Comparative 5% glycerine” varies greatly, from 1560 cps for the vanilla parfum to 14600 cps for the mango parfum, whereas the viscosity of the composition according to the invention remains in conformity with the desired viscosity, with a viscosity of less than 2500 cps, whatever the parfum used. The composition as described in Table 9 thus has a flow capacity and rheofluidification in accordance with the technical effect sought according to the invention.

1. A surfactant composition comprising:
a) at least one anionic surfactant, and
b) at least one compound chosen from amphoteric surfactants and non-ionic surfactants,
wherein the total active material of said surfactants is between 25 and 70% by weight relative to the total weight of said composition,
wherein the anionic surfactant ratio is between 0.2 and 0.9, the amphoteric surfactant ratio is between 0.0 and 0.8 and the non-ionic surfactant ratio is between 0.0 and 0.8, said ratio being the ratio of the percentage by weight of active material of said surfactant in said composition to the percentage by weight of total active material of said surfactants in said composition,
the pH of the composition being between 4 and 9,
wherein said at least one anionic surfactant is an alkyl sulfoacetate, an isethionate and/or a sarcosinate.

2. The composition according to claim 1, wherein said anionic surfactant is at least one alkyl sulfoacetate.

3. The composition according to claim 1, wherein said alkyl sulfoacetate is sodium lauryl sulfoacetate, sodium coco sulfoacetate or myristyl sulfoacetate.

4. (canceled)

5. The composition according to claim 1, wherein said sarcosinate is sodium lauroyl sarcosinate, sodium myristoyl sarcosinate, potassium myristoyl sarcosinate, sodium cocoyl sarcosinate, sodium oleyl sarcosinate, ammonium oleyl sarcosinate or triethanolamine lauroyl sarcosinate.

6. (canceled)

7. The composition according to claim 1, wherein said isethionate is selected from the group consisting of sodium cocoyl isethionate sodium lauroyl methyl isethionate and mixtures thereof.

8. (canceled)

9. The composition according to claim 1, comprising an alkyl sulfoacetate and at least one anionic surfactant chosen from sodium lauroyl methyl isethionate and/or sodium lauroyl sarcosinate.

10. (canceled)

11. The composition according to claim 1, wherein said at least one amphoteric surfactant is chosen from N-alkylamidobetaines, betaines, sultaines, alkylpolyaminocarboxylates, alkylamphoacetates, derivatives thereof, and/or glycine derivatives.

12. (canceled)

13. The composition according to claim 1, wherein said at least one non-ionic surfactant is chosen from glycolipids, alkypolyglucosides, glyceryl and fatty acid esters, sucrose and fatty acid esters, oxyalkylene sucrose esters, oxyalkylene glycerol esters, polyethylene glycol and fatty acid esters, sorbitan and fatty acid esters, polyglycerol fatty alcohols and/or glucamine derivatives.

14-15. (canceled)

16. The composition according to claim 1, wherein said amphoteric surfactant is cocamidopropylbetaine or sodium cocoamphoacetate, and the non-ionic surfactant is decylglucoside.

17-18. (canceled)

19. The composition according to claim 1, comprising an anionic surfactant consisting of sodium lauroyl methyl isethionate, an amphoteric surfactant consisting of cocamidopropyl betaine and a non-ionic surfactant consisting of decylglucoside.

20. (canceled)

21. The composition according to claim 1, wherein said surfactant composition is free of sulphated surfactant and/or of sodium chloride other than that which may be provided by the surfactants.

22-23. (canceled)

24. The composition according to claim 1, further comprising from 0.0 to 8% of sodium chloride other than that which may be provided by the surfactants, the value of 0.0% being excluded.

25. A cosmetic or dermatological composition comprising the surfactant composition according to claim 1, wherein said composition is a shower gel, facial skin cleansing gel, shampoo or hand washing gel.

26. (canceled)

27. A surface cleaning detergent composition comprising the surfactant composition as according to claim 1.

28. The detergent composition according to claim 27, wherein said detergent composition is a laundry detergent, a dishwashing liquid or a surface cleaner.

29. A method for cleansing skin and/or skin appendages comprising applying the cosmetic composition according to claim 25 to skin and/or skin appendages.

30. A method of non-therapeutic cosmetic treatment comprising applying to the skin and/or skin appendages and/or scalp the surfactant composition according to claim 1.

31. A combination of a spray-generating device and a surfactant composition according to claim 1, wherein said spray-generating device is capable of spraying said cosmetic composition.

32. A method of non-therapeutic cosmetic treatment comprising applying to the skin and/or skin appendages and/or scalp the cosmetic composition according to claims 25.

33. A combination of a spray-generating device and the cosmetic composition according to claim 25, wherein said spray-generating device is capable of spraying said cosmetic composition.
